(19) United States
(12) Patent Application Publication
(10) Pub. No.: US 2012/0016312 A1
(43) Pub. Date: Jan. 19, 2012

(54) CATHETER STABILIZATION DEVICE

(76) Inventors: Henry J.H. Brown, Ann Arbor, MI (US); Adrienne Rose Harris, Tecumseh, MI (US); Steven B. White, Ann Arbor, MI (US); Elyse Kemmerer, Ann Arbor, MI (US); Rebecca Ann Lehrmann, Ann Arbor, MI (US); Benjamin Lee Pierce, Rives Junction, MI (US)

(21) Appl. No.: 13/111,889

(22) Filed: May 19, 2011

Related U.S. Application Data
(60) Provisional application No. 61/346,285, filed on May 19, 2010.
(60) Provisional application No. 61/356,784, filed on Jun. 21, 2010.

(51) Int. Cl. A61M 25/02

(52) U.S. Cl. 604/180; 604/174

(57) ABSTRACT

A catheter stabilization device for stabilizing an inserted catheter penetrating a surface at a catheter insertion site, including: a base that is coupleable to the surface and includes a channel defining a longitudinal opening that receives a portion of the catheter, and a frame that forms at least a partial perimeter around the catheter insertion site with a plurality of anchoring points; and a catheter fitting, coupled to the base, that secures the catheter in the channel. The base couples to the surface at the plurality of anchoring points distributed around the catheter insertion site, thereby stabilizing the catheter. The method of using the device includes positioning the base over the catheter, receiving a portion of the catheter in the channel through the longitudinal opening, securing the catheter within the channel, and coupling the base to the surface at the plurality of anchoring points distributed around the insertion site.

### CROSS-REFERENCE TO RELATED APPLICATIONS

This application claims the benefit of U.S. Provisional Application Nos. 61/346,285 filed 19-May-2010 and 61/356,784 filed 21-Jun.-2010, which are each incorporated in its entirety by this reference.

### TECHNICAL FIELD

This invention relates generally to the intravenous therapy field, and more specifically to a new and useful catheter stabilization device in the intravenous therapy field.

### BACKGROUND

Patients undergoing medical treatment often require a form of intravenous (IV) therapy, in which a fluid is administered to the patient through a vein of the patient. IV therapy is among the fastest ways to deliver fluids and medications into the body of the patient. Intravenously infused fluids, which typically include saline, drugs, blood, and/or antibiotics, are conventionally introduced to the patient through a flexible catheter positioned at any of several venous routes, such as peripheral veins and central veins.

To set up IV therapy with conventional devices and methods, the caregiver positions the catheter over the selected vein and uses a needle within the catheter to pierce the skin and allow insertion of the distal end of the catheter into the vein. The proximal end of the catheter, relative to the midline of the catheter, is fixed to the end of a catheter hub that is proximal relative to the midline of the patient. The caregiver connects the catheter to a fluid supply through external tubing, including extension tubing that is typically attached to the catheter hub and that the caregiver typically bends into a U-shape to accommodate the typical opposite positions of the catheter and IV fluid source. To avoid unscheduled IV line restarts, the catheter and tubing are typically secured against the skin of the patient with tape or similar catheter stabilization devices (CSDs) such as adhesive stabilizing pads that restrain the catheter hub.

However, these conventional devices and methods for IV therapy have drawbacks. The extension tubing may catch on nearby obstacles during patient movement or caregiver manipulation, which may cause painful vein irritation and comprise the IV. Tape and other existing CSDs are not optimal for stabilization because securing the round, rigid, and bulky components such as the catheter and tubing against relative flat skin can be difficult and ineffective. Tape and other existing CSDs do not fully prevent the catheter from moving within the vein, which leads to patient-endangering complications including catheter dislodgement, infiltration (fluid entering surrounding tissue instead of the vein) and phlebitis (inflammation of the vein). Adhesive stabilizing pads tend to result in other undesired effects, such as skin irritation and/or breakdown due to prolonged concentrated adhesion to the skin. Furthermore, tape and current CSDs do not prevent the catheter from painfully and dangerously pivoting around the insertion site and moving within the vein.

Thus, there is a need in the intravenous therapy field to create an improved catheter stabilization device that overcomes one or more of the drawbacks of conventional vascular delivery systems. This invention provides such an improved catheter stabilization device.

### BRIEF DESCRIPTION OF THE FIGURES

FIGS. 1A and 1B are a perspective view and transparent schematic, respectively, of the catheter stabilization device of a preferred embodiment;

FIG. 2 is a top view schematic of the catheter stabilization device of a preferred embodiment;

FIGS. 3A and 3B are perspective and top view schematics of a variation of the catheter stabilization device of a preferred embodiment;

FIG. 4A is a cross-sectional view of the catheter stabilization device of a preferred embodiment;

FIG. 4B is a cross-sectional view taken along line A-A of FIG. 4A, of the catheter stabilization device of a preferred embodiment;

FIGS. 5B-5D are cross-sectional views of variations of the catheter fitting taken along line B-B of FIG. 5A;

FIG. 6 is a schematic of the plurality of anchoring points in the catheter stabilization device of a preferred embodiment;

FIGS. 7A-7C are schematics of variations of the frame portion of the base in the catheter stabilization device of a preferred embodiment;

FIGS. 8A-8C and 9A-9B are schematics of variations of the base in the catheter stabilization device of a preferred embodiment;

FIGS. 10A-10D are top, side, front, and perspective views, respectively, of an alternative embodiment of the base in the catheter stabilization device;

FIGS. 11B and 11C are cross-sectional views of the unlocked and locked modes of a catheter fitting, respectively, taken along the line C-C of FIG. 11A, in the catheter stabilization device of a preferred embodiment;

FIGS. 12A and 12B are schematics of a variation of the catheter fitting in the catheter stabilization device of a preferred embodiment;

FIGS. 13A-13B and 14A-14B are schematics of variations of the attachment element of the catheter stabilization device of a preferred embodiment;

FIGS. 15A-15D are top view, cross-sectional view taken along the line D-D of FIG. 15A, front view, and side view, respectively, of the catheter stabilization device of a preferred embodiment; and

FIGS. 16A-16F are illustrations of a method of stabilizing an inserted catheter penetrating a surface.

### DESCRIPTION OF THE PREFERRED EMBODIMENTS

The following description of preferred embodiments of the invention is not intended to limit the invention to these preferred embodiments, but rather to enable any person skilled in the art to make and use this invention.

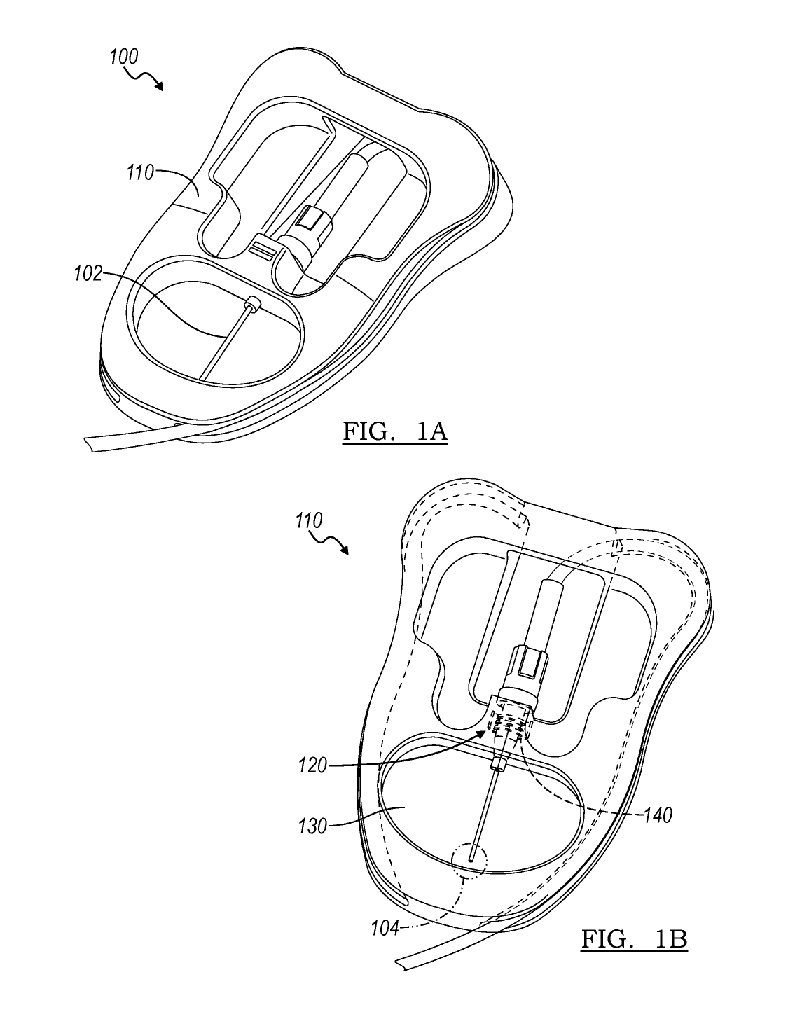

As shown in FIGS. 1 and 2, the catheter stabilization device 100 of a preferred embodiment includes: a base no that is coupleable to the surface and includes a channel 120 having a longitudinal opening 122 that receives a portion of the catheter 102 and a frame 130 that forms at least a partial perimeter around the catheter insertion site 104 with a plurality of anchoring points, in which the base no couples to the surface at the plurality of anchoring points; and a catheter fitting 140, coupled to the base, that selectively secures the catheter 102 in the channel 120. In some embodiments, an attachment element 160, integrated with the catheter stabilization device 100 or part of an external attachment element, couples the base no to the surface. In some embodiments, the catheter stabilization device may include a tubing router 150 that secures and routes tubing 106 that is coupled in fluid communication with the catheter, preferably routing the tubing through a 180-degree turn 154. The base 110 may include two or more separable portions, such that a second portion is removably coupled to a first portion to facilitate modularity that enables the system to be used in a variety of locations on the body with different space and movement characteristics.

The catheter stabilization device 100 preferably securely and safely stabilizes a catheter that has been inserted into a patient to provide intravenous (IV) access through the catheter to a vein or other blood vessel of the patient undergoing IV therapy or any other catheter-based therapy, although in an alternative embodiment the catheter stabilization device may be coupled to the catheter prior to catheter insertion. The catheter stabilization device is preferably used to stabilize and maintain an W line on a peripheral vein such as on the arm, hand or leg, but may alternatively be used for central or peripheral venous access on the neck, chest, abdomen, any suitable intravenous or intraarterial location, or any suitable surface or catheter location. By reducing relative movement between the catheter and vessel in which the catheter is inserted, the catheter stabilization device preferably reduces complications such as pain, catheter dislodgement, infiltration, and phlebitis, thereby improving patient comfort and patient care.

As shown in FIG. 2, the base 110 of the catheter stabilization device functions to receive a catheter 102 penetrating patient skin or other suitable surface at an insertion site 104, and to stabilize the catheter on the skin with a plurality of anchoring points distributed around the catheter insertion site. However, in an alternative embodiment, the base no may receive or otherwise be coupled to the catheter 102 prior to catheter insertion into the patient or other surface, and with a predetermined or adjustable position of the catheter within the base. For example, in some versions of the system, the medical practitioner may insert the catheter into the base prior to establishing an W line in the patient. In other versions of the system, the catheter may be integrated within the channel 120 as part of the base such as during pre-assembly in manufacturing, to provide an integrated system that is cost-efficient, reduces W setup time, and reduces packaging waste.

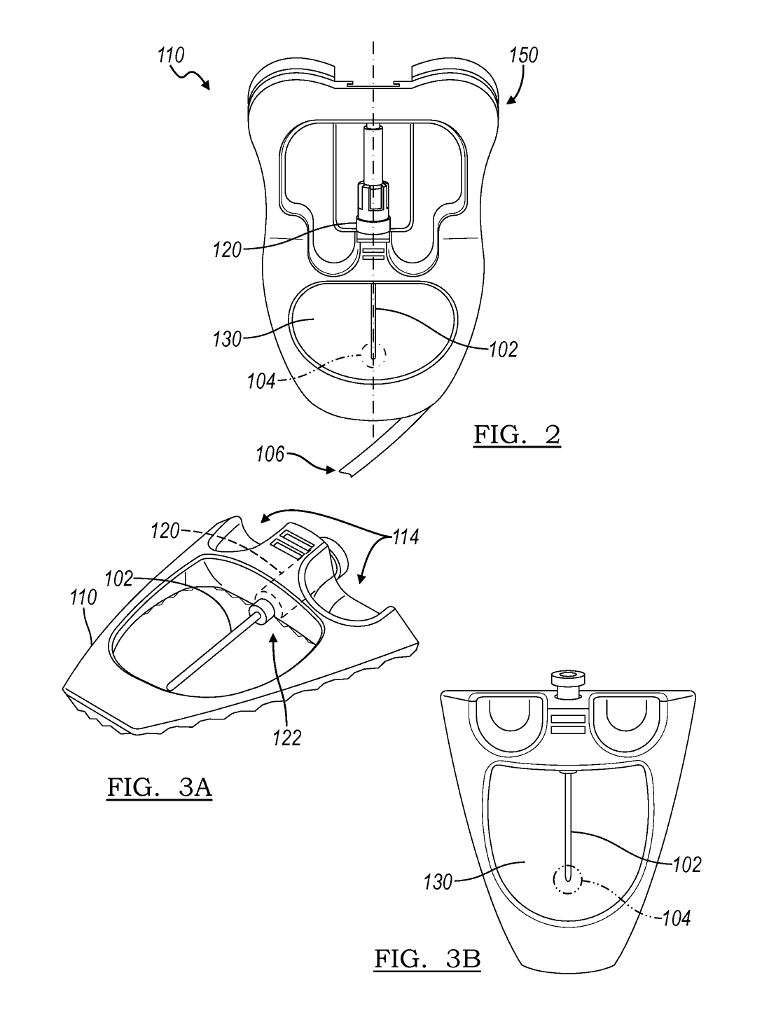

In one embodiment, as shown in FIGS. 3A and 3B, the overall profile of the base 110 is relatively thin and/or wide, which may decrease the risk of the base catching or snagging on bed equipment or other obstacles, and help distribute forces over a greater area of the skin which may lower the chances of the patient developing skin irritations, sores, and other degradations. Furthermore, the surface of the base no that contacts the skin may be substantially flat, slightly curved, or customized to conform to a particular surface, such as an underside concavity to conform to a limb, finger, or knuckle or other body surface. The base no preferably includes a channel 120 with a longitudinal opening 122 that is configured to receive at least a portion of the catheter 102, and a frame 130 that forms at least a partial perimeter around the catheter insertion site 104 with a plurality of anchoring points 132. Although in a preferred embodiment the base 110 includes one channel with one longitudinal opening and one frame, the base may alternatively include multiple channels, multiple longitudinal openings, and/or multiple frames to enable stabilization of multiple catheters and/or flexibility in positioning the base relative to a particular catheter location.

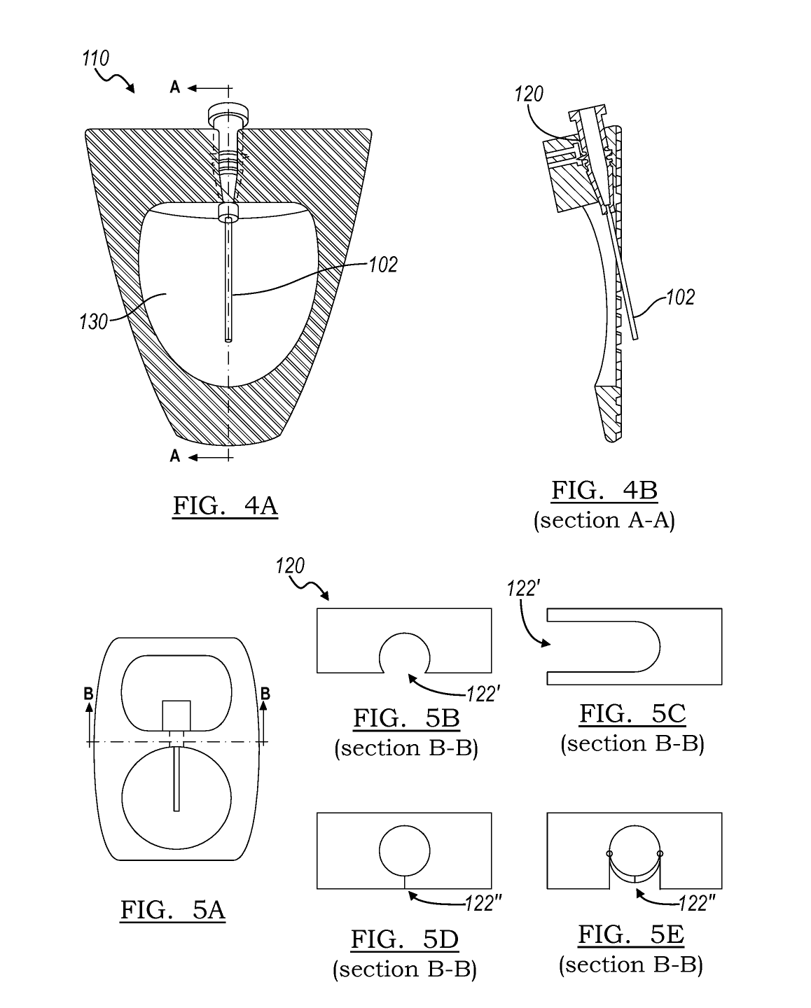

As shown in FIGS. 3-5, the channel 120 is preferably defined by the underside of the base no, such that the base may be placed over an inserted catheter (such as by approaching the catheter in a downward direction towards the skin) to receive the catheter in the channel in the underside of the base. However, the channel 120 additionally and/or alternatively may be at least partially defined by a lateral side, upper side, and/or any suitable side of the base, such that the channel may receive the catheter when the base approaches the catheter in a lateral, upward, and/or any suitable direction. The channel 120 may also alternatively receive the catheter in a longitudinal direction, such that the catheter longitudinally slides into the catheter. The channel may have a cross-section of any suitable arc segment of a circular or non-circular ellipse, a rectangular cross-section or other polygonal shape, or any suitable shape, such as a custom channel to complement the cross-sectional shape of a particular catheter hub. The channel 120 may be angled or non-parallel with respect to an external surface, such as the underside, of the base. For example, the channel may be angled such that when the catheter is in the channel, the inserted distal end of the catheter is oriented closer to the skin than the opposite proximal end of the catheter. Alternatively, the channel may be within a pivotable piece that allows the catheter to be at an adjustable angle of insertion into the insertion site. The pivotable piece may be lockable at a desired angle relative to the skin by a pin or other mechanism to permanently or temporarily fix the catheter in a particular angular position relative to the skin.

As shown in FIG. 5, the channel 120 preferably defines a longitudinal opening 122 through which the catheter is received in the channel. Similar to the channel location, the longitudinal opening 122 is preferably defined by the underside of the channel, but additionally and/or alternatively may be at least partially defined by a lateral side, upper side, and/or any suitable side of the channel. In a first variation, as shown in FIGS. 5B and 5C, the longitudinal opening 122′ is an open slot, such that the channel 120 has a non-enclosed cross-section. In this variation, the channel may have any suitable non-enclosed cross-section with a partial perimeter or open arc segment (e.g., semi-circular), such that the channel 120 only partially surrounds the entire circumference of at least a portion of the catheter and the catheter is received into the channel through the open arc segment. In a second variation, as shown in FIGS. 5D and 5E, the longitudinal opening 122″ is a slit, such that the channel 120 has an enclosed cross-section that surrounds the entire circumference of at least a portion of the catheter, and the channel is openable at the slit 122″. For example, on one or both sides of the slit, the channel 120 may include compressible material (e.g. elastomer, foam) that compresses to admit insertion of the catheter through the slit into the channel. As another example, one or both edges of the slit may retract or swing open (e.g. with grips, levers, switches, buttons, or another suitable mechanism) to admit insertion of the catheter through the slit into the channel. In this second variation, the slit may initially be open or closed before the channel 120 receives the catheter, and may return to its initial open or closed position after the channel receives the catheter. For example, the slit may initially be open, but may close after the channel receives the catheter. In an alternative embodiment, the channel may lack a longitudinal opening, such that the catheter longitudinally slides into the channel.

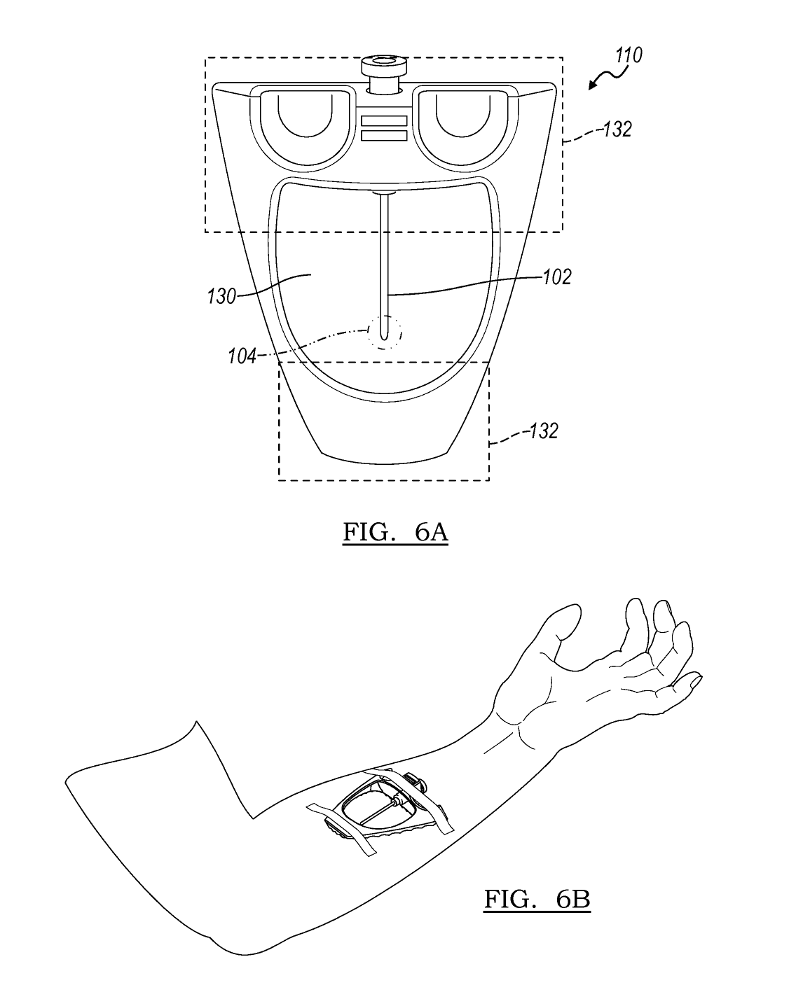

The frame 130 of the base no is configured to form at least a partial perimeter around the catheter insertion site with a plurality of anchoring points 132. The frame 130 preferably provides access to the catheter insertion site 104, such as for IV setup and/or maintenance and visual access for monitoring. The frame 130 provides anchoring points are distributed around the insertion site, with at least two anchoring points 132 that are coupleable to the surface and substantially oppose one another across the insertion site (e.g., proximal and distal sides, or opposite lateral sides of the insertion site), or at least three anchoring points distributed around the insertion site. The plurality of anchoring points are preferably approximately equally distributed around the insertion site, but may alternatively be unequally distributed around the insertion site. When secured to the patient, the frame 130 preferably restricts motion of the catheter relative to the insertion site, thereby significantly reducing the number of degrees of freedom of the catheter, including pivoting, rotating, and translation both horizontally in a lateral plane and vertically. Furthermore, as shown in FIG. 6, in a common use scenario in which the catheter is aligned in a distal-proximal direction (relative to the on the forearm of a patient with the insertion site near the inside of the elbow, the secured frame 130 further stabilizes the catheter by causing the catheter to be pulled further into the blood vessel if the extension tubing, IV supply bag, or other catheter-related tubing is pulled or caught on an object such as the bed frame.

In a first preferred embodiment, as shown in FIG. 1-3, the frame 130 includes an enclosed perimeter configured to surround the catheter insertion site 104, which may provide more surface area contact with the patient for potentially increased securement and distributed stress on the skin, and stabilizes the catheter in multiple directions. The frame 130 may be substantially elliptical, providing enough space around the insertion site to reduce skin irritation near the insertion site and provide easy access to the insertion site. The frame may alternatively be radially symmetrical about the insertion site. However, the enclosed frame may be any suitable shape. In one variation, the frame provides an anchoring point on at least two opposite sides of the insertion site, such as on opposite sides that are in line with the received catheter and/or transverse to the received catheter. For example, for a common use scenario as shown in FIG. 6, typically the catheter may be aligned in a distal-proximal direction (relative to the midline of the patient) when inserted in a patient, the frame is preferably shaped such that part of the frame lies proximal to the insertion site and another part of the frame lies distal to the insertion site, which reduces lateral movement such as pivoting about the insertion site.

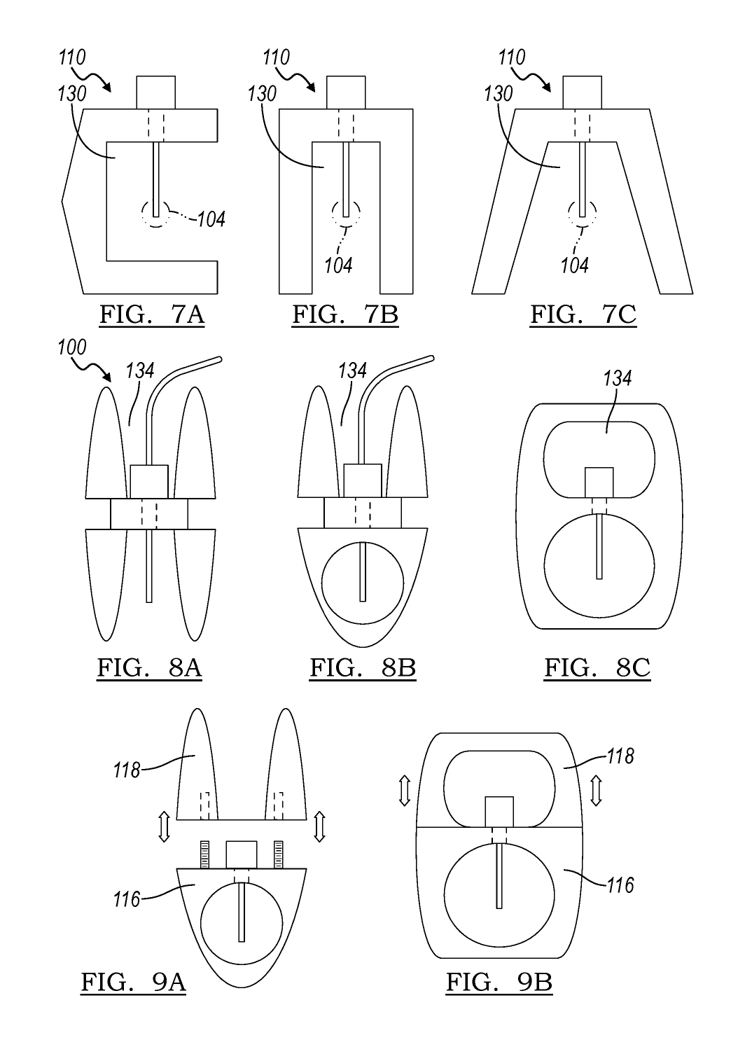

In a second preferred embodiment, the frame 130 includes a partial perimeter that provides at least two anchoring points by which the base 110 couples to the skin. For example, the frame 130 may be generally C-shaped or U-shaped, with an opening on at least one side. As shown in FIG. 7A, in a first variation of this embodiment, the partial frame surrounds the insertion site 104 on at least proximal and distal sides of the insertion site, with an opening of the frame lying at least partially lateral to the insertion site. As shown in FIGS. 7B and 7C, in a second variation of this embodiment, the partial frame 130 surrounds the insertion site on at least two sides that are lateral to the catheter, with an opening of the frame lying distal to the catheter tip (relative to the midline of the catheter). However, the anchoring points may be on any suitable sides of the insertion site. In particular, the partial frame may include three or more anchoring points of stabilization (e.g. the end points of two frame legs and the point of contact where the catheter is secured in the channel of the base). The three or more anchoring points of stabilization may be radially symmetrically distributed about the insertion site, to more equally distribute forces around the insertion site, or may be radially asymmetrically distributed.

In some applications, the enclosed frame 130 of the first preferred embodiment may provide greater rigidity and stability of the catheter. In some other applications, the partial frame of the second preferred embodiment may reduce torsional rigidity of the base, thereby increasing the ability of the frame to conform to curved or other nonplanar body surfaces to provide catheter stabilization on body surfaces of a complex curve (e.g., body surfaces bending along two or more non-parallel axes). However, each embodiment of the frame may be used for any suitable application.

As shown in FIG. 3A, the base no may further include a recess 114 on one or more sides of the channel 120, which may provide a grip for the fingers of a medical practitioner or other user to handle the base, such as during connecting extension tubing to the catheter or removal of the catheter from the patient.

In some embodiments, the base no may include at least two portions 116 and 118. Preferably the two portions include a distal portion 116 that incorporates the frame 130 and/or the channel, and a proximal portion 118 that also couples to the skin to provide additional support for the catheter around the insertion site, although the base may be divided into any suitable portions. Another portion of the base 110 may include a second frame 134 that increases access to extension tubing and other tubing associated with the catheter. For example, the proximal portion may include a second frame 134 similar to the first frame 130, such as a partial frame (e.g., with legs shown in FIGS. 8A and 8B) or an enclosed frame (e.g., as shown in FIG. 8C). As shown in FIGS. 9A and 9B, a second portion 118 may be removably coupled to a first portion 116, such as to facilitate modularity that enables the system to be used in a variety of locations on the body with different space and movement characteristics. For example, more than one portion of the base may be coupled to one another to enable securement of the catheter on a body part with relatively large surface area (e.g., arm), while one portion of the base may be removed from the system to enable securement of the catheter on a body part with a relatively small surface area (e.g., wrist). The base portions may be removably coupled with a mechanism such as screw threads (FIG. 9A), slots and tabs, a snap fit, latches, screws or other fasteners, adhesive, or any suitable mechanism.

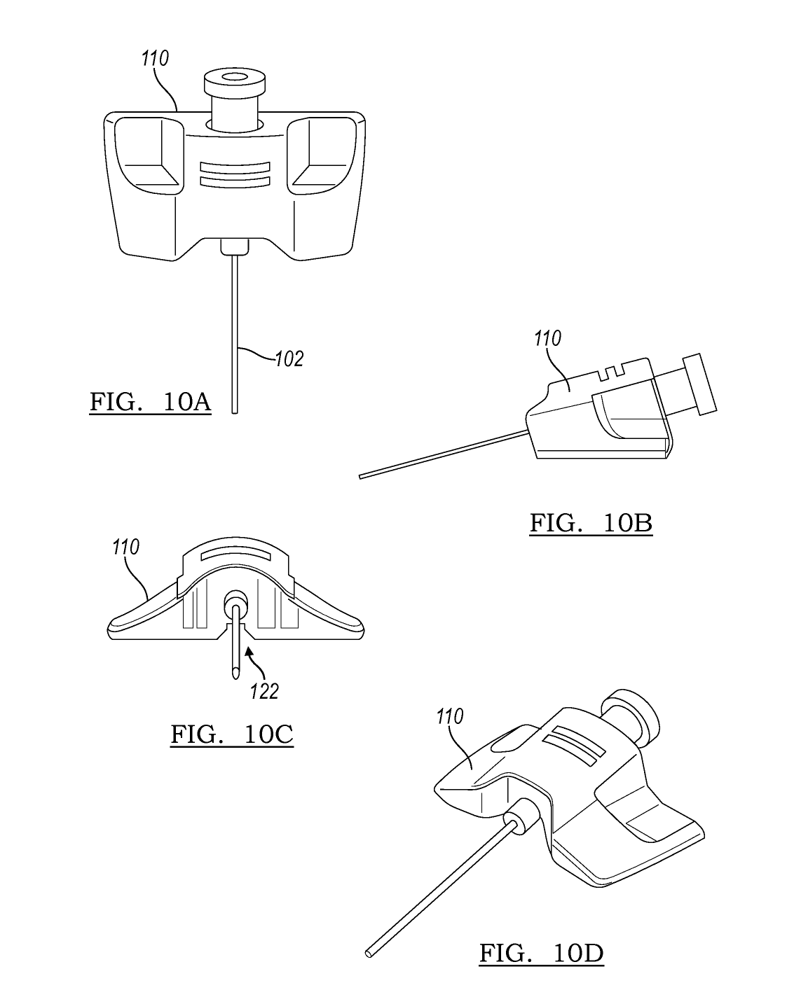

In some alternative embodiments, as shown in FIGS. 10A-10D, the base 110 may include a channel 120 that receives a catheter as described above, but may lack a frame forming a perimeter. In these alternative embodiments, the base provides an anchoring point at least on one side of the catheter insertion site 104.

The base 110 is preferably made of a flexible, semi-rigid material such as silicone that provides structural support to the system, yet is compliant and can more easily conform to body surfaces, particularly complex contoured body surfaces. The base may additionally and/or alternatively include a hard plastic like nylon, or any polymer, metal, composite, or other suitable material. For example, a portion of the frame 130 of the base at least partially surrounding the insertion site may be made of a more flexible material, while the portion of the base defining the channel that receives the catheter may be made of a more rigid material. The base is preferably made by injection molding, but may be formed through stereolithography, casting, milling, and/or any suitable manufacturing process.

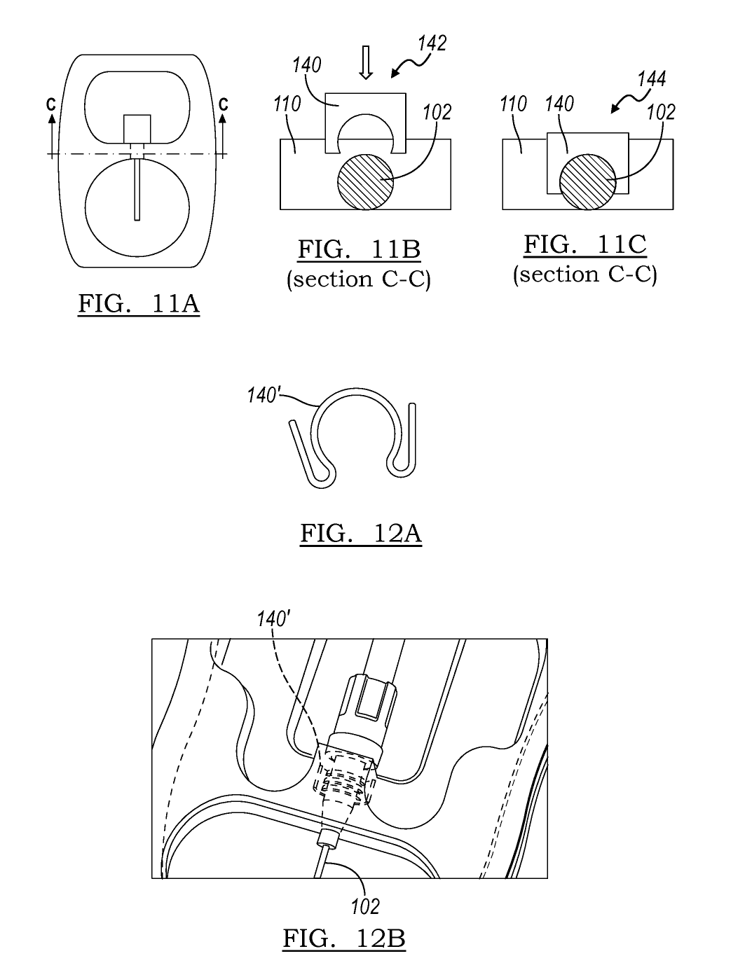

The catheter fitting 140 of the catheter stabilization device functions to secure the catheter in the channel 120 of the base 110. The catheter fitting may be impermanently or permanently secure the catheter, such that once the catheter is inserted into the channel 120, the catheter may be removable such as for swapping catheters, or may be permanently positioned in the channel 120 such that removal of the catheter entails removal of the base. The catheter fitting is preferably coupled to the base, such as to the channel 120 or any suitable portion of the base proximate to where the catheter is received. The catheter fitting may additionally and/or alternatively be integrated into the channel. The catheter fitting may include one or more of several variations. As shown in FIG. 11, in a first variation, the catheter fitting is engaged with the base 110 such that the catheter selectively operates between an unlocked position 142 (FIG. 11B) in which the catheter fitting 140 does not secure the catheter in the channel, and a locked position (FIG. 11C) in which the catheter fitting 140 secures the catheter in the channel. The catheter fitting 140 may include a button, switch or other toggling mechanism that the user operates to manipulate the catheter fitting between the unlocked and locked positions. In this first variation, the catheter fitting may be formed such that in the locked position, the catheter fitting conforms to the received portion of the catheter. For example, the catheter fitting may include a sleeve or partial circumferential fitting that snap fits with the received catheter (e.g. catheter hub or other proximal portion of the catheter) in the locked position, thereby securing the catheter in the channel. As another example, the catheter fitting may additionally and/or alternatively include compressible material such as foam or rubber that compresses to conform to the received catheter. The catheter fitting may be slidingly engaged with the base with slots, grooves, an interference fit, or other suitable mutual articulations, or engage with the base in any suitable manner between the unlocked and locked positions. The fitting 140 may further include adhesive or other attractive forces to help retain the catheter within the channel 120.

As shown in FIG. 12, in a second variation, the catheter fitting is substantially fixed within or otherwise coupled to the channel 120 of the base 110. For example, the catheter fitting may include a flexible fitting 140′ such as a spring clip (FIG. 12A) that flexes to accept a portion of the catheter and restores (e.g. in a snap fit or press fit) to secure the catheter. As shown in FIG. 12B, the flexible fitting includes an arcuate portion or other suitable cross-section that receives the catheter 102, and may include lateral wings that are adapted to help retain the spring clip in the channel. The flexible fitting 140′ may include features such as slots to reduce rigidity and improve flexibility without significantly reducing retaining strength. However, any suitable flexible fitting may be used. The flexible fitting may further include adhesive or other attractive forces to help retain the catheter within the channel 120.

Further variations of the catheter fitting may include combinations of the above variations, and may additionally and/or alternatively include other catheter retaining features such as internal threads that mate with external threads of the catheter hub, frictional bumps or grooves, a flange or other guide to help seat the catheter within the channel, and/or feedback mechanisms to indicate to the user confirmation of proper securement of the catheter within the catheter fitting and base. The feedback may be tactile (such as with the snap fit or latch), audible (such as a click of the catheter snapping into the catheter fitting), and/or visual (such as translucent material allowing the user to view seating of the catheter within the catheter fitting).

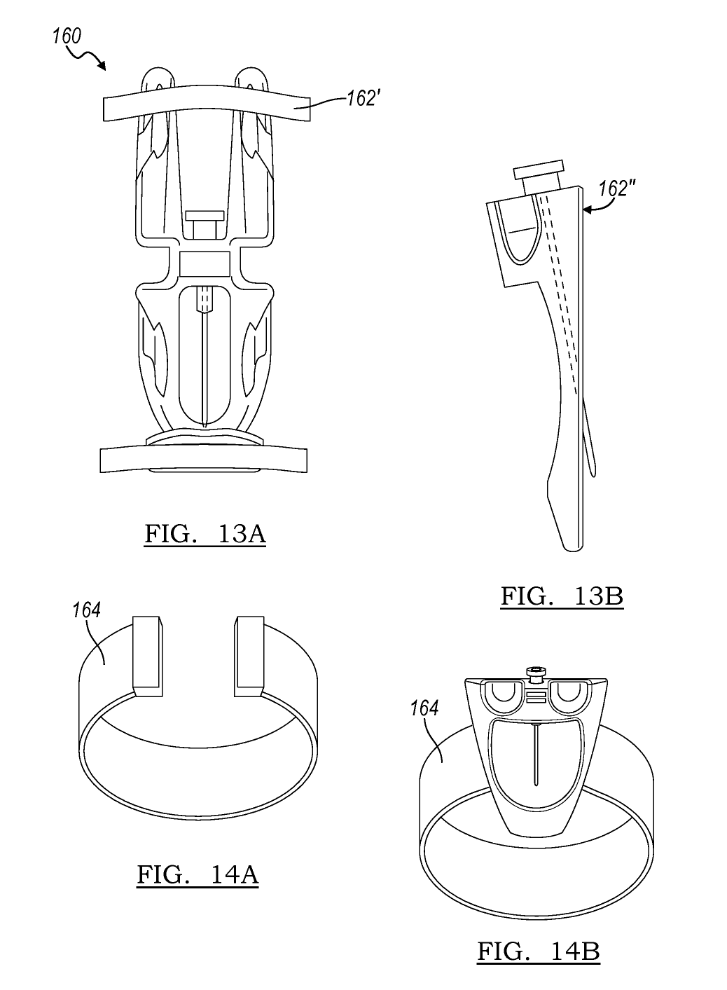

In some embodiments, the catheter stabilization device further includes an attachment element 160 and/or interfaces with a separate attachment element. The attachment element 160 of the catheter stabilization device functions to couple the base 110 to the surface, thereby securing the received catheter 102 relative to the insertion site 104. The attachment element 160 may be one or more of several variations. In a first variation, as shown in FIG. 13, the attachment element 160 includes adhesive 162 applied to the base 110 and/or to the skin of the patient. For example, the attachment element 160 may include tape that passes over a portion of the base and straps the base onto the skin. In particular, tape 162′ may be applied to the base 110 and skin in a lateral direction across the insertion site and perpendicular to the catheter, but additionally and/or alternatively in a longitudinal direction aligned with the catheter, or in any suitable direction. In some embodiments, as shown in FIG. 13A, the tape may pass over a designated taping surface with increased surface area for greater attachment strength. In another example of the first variation, as shown in FIG. 13B, the attachment element 160 may include adhesive 162″ coupled to a surface of the base 110 (e.g. underside or other side to be placed in contact with the skin). The adhesive may initially be covered with a protective cover, such the protective cover is removed to expose adhesive that couples the base to the surface, or may be activated by temperature or any suitable trigger.

In a second variation, as shown in FIG. 14A and 14B, the attachment element 160 is a strap 164 or cuff that is coupled to the base 110 and wraps around an appendage (e.g. arm, leg, or finger) of the patient or straps the base 110 to the patient in any suitable manner. In this variation, the strap 164 may pass over and/or under the base. For example, the base may couple to a strap 164 such as with adhesive or hook and loop fastener, or include a raised portion that securely mates with a portion of the strap, such as with a dovetail joint or latch. However, the attachment element 160 may be any suitable mechanism, substance, or structure that couples the base to the patient.

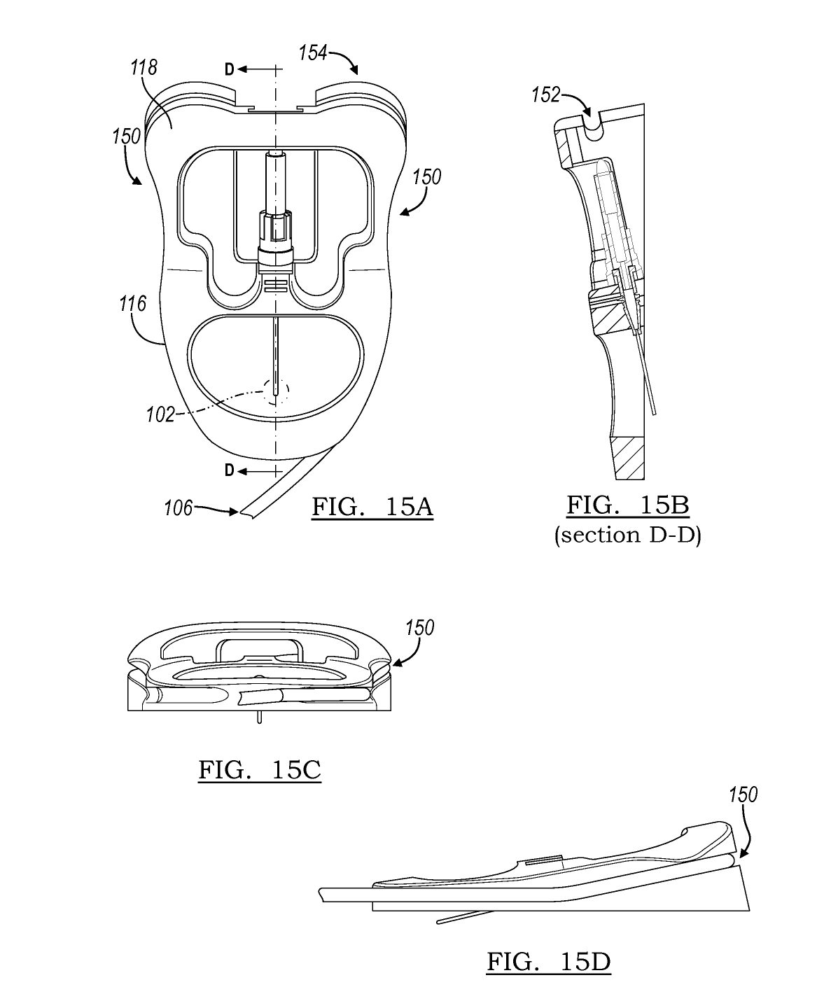

In some embodiments, the catheter stabilization device further includes a tubing router 150. The tubing router 150 functions to secure and route extension tubing 106 and/or other tubing associated with the catheter. As shown in FIGS. 15A-15D, the tubing router 150 preferably includes an approximately 180-degree turn 154 that routes the tubing from the proximal end of the catheter, around the base 110, and back towards the distal end of the catheter 102. In a typical IV treatment scenario, the approximately 180-degree loop securely and safely redirects extension tubing from the catheter towards a fluid supply, which is commonly placed near the head of a bed or otherwise proximal to the insertion site (relative to the midline of the patient). However, the tubing router 150 may include a loop of any suitable angular sweep, or may secure and redirect tubing in any suitable manner. The tubing router 150 also helps avoid dangers due to the extension tubing snagging on nearby obstacles and causing the inserted catheter to move relative to the insertion site. The tubing router may be on a removable portion of the base, which allows the tubing router to be an optional feature on the system.

In a preferred embodiment, the tubing router 150 may include a channel 152 or other recess that runs along at least one side of the base no. In one embodiment, as shown in FIGS. 15A-15D, the tubing router 150 includes two lateral channels: one that traverses a left side of the base, and another that traverses a right side of the base, which provides for flexibility in routing. For example, if the tubing router includes both left-side and right-side channels, the extension tubing may be routed on a particular side that is preferred in a given application, based on factors such as whether the IV fluid supply bag is on a left side or a right side of the patient, whether the catheter stabilization system is placed on a left side or a right side of the patient, or whether any obstacles on a particular side of the patient may interfere with the extension tubing. Alternatively, the tubing router 150 may include a channel or recess on only a left side or right side of the base no, or may be on an upper side, underside, or any suitable side of the base. The tubing router may be sized to allow the extension tubing to snap fit or be slightly compressed into the channel for suitable retention within the catheter, such as having a diameter slightly smaller than the diameter of the catheter, and/or having a cross-sectional arc length that surrounds at least half of the full circumference of the extension tubing. The tubing router 150 may be deep enough to enable the extension tubing to be seated somewhat recessed, such as for increased retention within the tubing router and reduced chance of snagging on an obstacle. In one variation, the tubing router 150 includes a compressible, deformable material such as foam, rubber, or putty to allow a range of tubing diameters to fit within the channel and/or to further increase retention of the tubing within the channel. In another variation, the tubing router additionally and/or alternatively includes clips, straps, tape, other adhesive and/or any suitable fasteners to help retain the tubing within the channel and/or along a suitable surface (e.g. upper side) of the base.

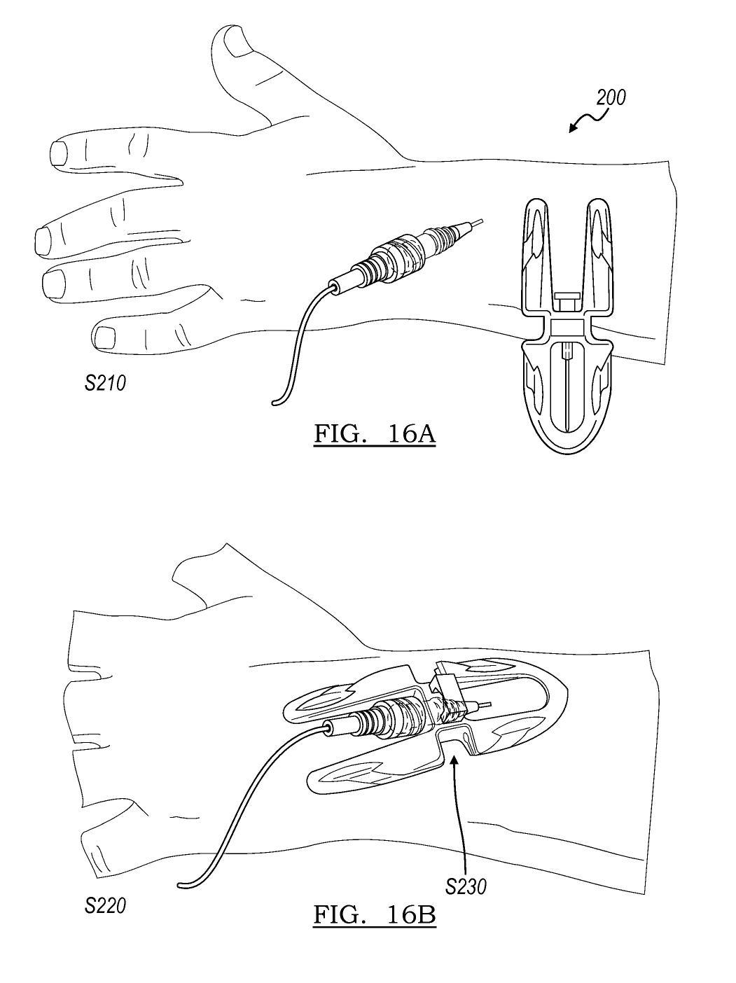

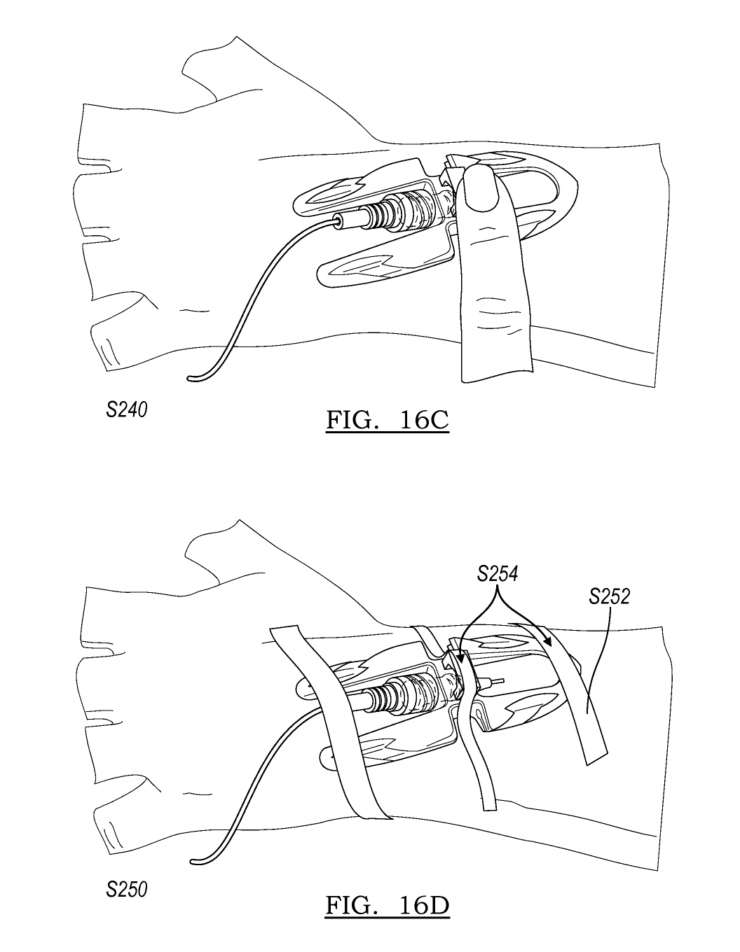

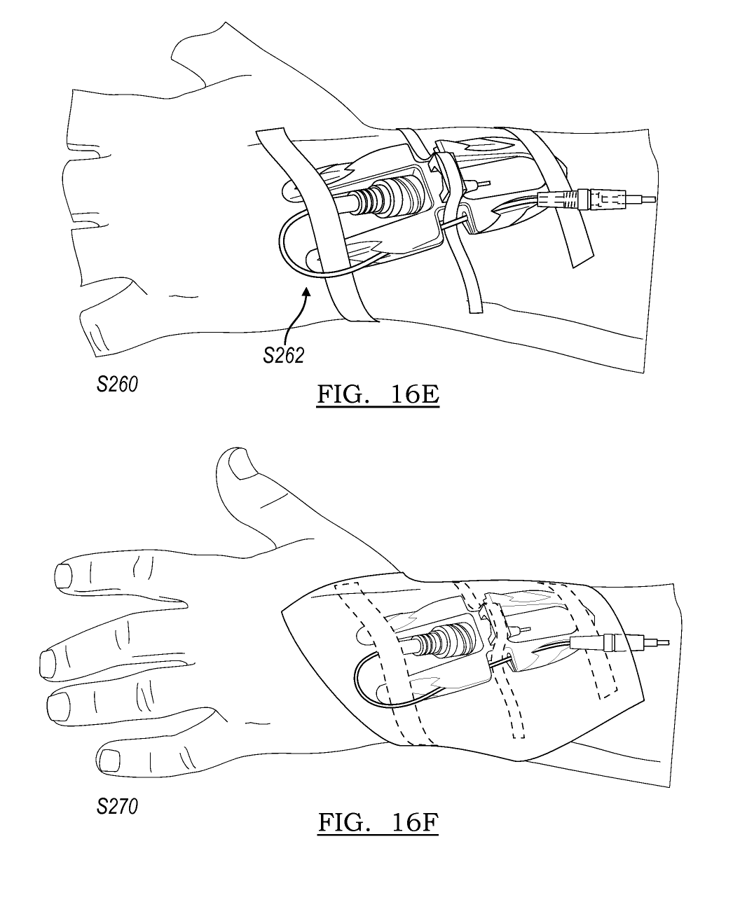

As shown in FIGS. 16A-16F, the method 200 for stabilizing an inserted catheter penetrating a surface at an insertion site of a preferred embodiment includes: providing a base S210 having an underside that defines a channel with a longitudinal opening; positioning the base over the catheter S220; receiving a portion of the catheter in the channel through the longitudinal opening S230; securing the catheter within the channel S240; and coupling the base to the surface at a plurality of anchoring points distributed around the insertion site S250.

As shown in FIG. 16A, providing a base S210 preferably includes providing a catheter stabilization device as described above. The catheter stabilization device is preferably one of any suitable variation described above. Alternatively, providing a base S210 includes providing any suitable base with a channel that is configured to receive a catheter.

Positioning the base over the catheter S220 functions to prepare the base to receive the catheter in the channel of the base. As shown in FIG. 16B, positioning the base preferably includes placing the base onto the catheter from an overhead direction relative to the surface. Alternatively, positioning the base may include placing the base onto the catheter from a lateral direction (e.g., from the left or right side of the catheter) or any suitable direction. The particular direction from which the base approaches the catheter may depend on the particular placement of the channel in the base, such as in which side of the base the channel is defined.

Receiving a portion of the catheter in the channel S230 and securing the catheter S240 jointly function to couple the base to the catheter. As shown in FIG. 16B, receiving a portion of the catheter S230 preferably includes receiving a catheter hub, but may alternatively include receiving any suitable part of the catheter, and/or tubing. As shown in FIG. 16C, securing the catheter S240 may include pressing a conformable fitting over the received portion of the catheter. The conformable fitting may be a movable fitting operable between an unlocked mode and a locked mode, or may be a flexible fitting such as a spring clip, or any suitable variation.

Coupling the base to the surface S250 at a plurality of anchoring points distributed around the insertion site functions to stabilize the received catheter relative to the insertion site. As shown in FIG. 16D, coupling the base to the surface S250 preferably includes coupling the base to the surface at two or more substantially opposing anchoring points S254. For example, coupling the base may include coupling the base to the surface at least at one anchoring point proximal to the insertion site and at another anchoring point distal to the insertion site. As another example, coupling the base may include coupling the base to the surface at least at two substantially opposing anchoring points lateral to the catheter site. The multiple anchoring points may, however, be located in any suitable equal or unequal distribution around the insertion site (e.g. three anchoring points distributed radially around the insertion site in approximately 60-degree increments).

Coupling the base to the surface may performed with any suitable attachment element. In one variation, as shown in FIG. 16D, coupling the base includes taping the base to the surface S252 (e.g. medical tape strips). In another variation, coupling the base includes applying adhesive on the underside (or other suitable surface) of the base to affix the base to the surface. In yet another variation, coupling the base includes applying a strap over the system and around the surface. Coupling the base to the surface may alternatively include coupling the base to an intermediary surface that in turn is coupled to the surface in which the catheter is inserted, such that the base is indirectly coupled to the surface in which the catheter is inserted. However, other variations of coupling the base to the system may be performed. Furthermore, coupling the base to the surface may further include conforming the base to the surface, such as by flexing the base. For example, portions of the base may be bent to complement the curvature of an arm of the patient.

As shown in FIG. 16E, the method may further include routing tubing associated with the catheter in a tubing router S260, which functions to safely and securely redirect tubing that may otherwise become snagged on nearby obstacles. The tubing may be extension tubing or any suitable tubing or structures that requires redirecting. Routing tubing S260 preferably includes routing tubing through an approximately 180-degree turn S262, although may additionally and/or alternatively include routing tubing in any suitable path or manner.

As shown in FIG. 16F, the method may further include applying a dressing over the insertion site S270 and/or at least a portion of the base, which functions to help protect the insertion site against bacteria, viruses, and other pathogens. The dressing may be a breathable, sterile dressing such as Tegaderm, which is known and used by one skilled in the art. The dressing is preferably transparent to allow visualization of the insertion site, and includes adhesive to attach to the surface. The dressing may further contribute to coupling the base to the surface. However, the dressing may additionally and/or alternatively include any suitable device or method to assist in the protection of the insertion site.

Preferred embodiments of the catheter stabilization device include every combination and permutation of the various aspects of the elements, including the base, channel, catheter fitting, tubing router, and attachment element. Furthermore, preferred embodiments of the method for stabilizing an inserted catheter include every combination and permutation of the various steps.

As a person skilled in the art will recognize from the previous detailed description and from the figures and claims, modifications and changes can be made to the preferred embodiments of the invention without departing from the scope of this invention defined in the following claims.

We claim:

1. A catheter stabilization device for stabilizing an inserted catheter penetrating a surface at a catheter insertion site, comprising:
a base that is coupleable to the surface, wherein the base includes:
a channel defining a longitudinal opening that receives a portion of the catheter; and
a frame that forms at least a partial perimeter around the catheter insertion site with a plurality of anchoring points;
wherein the base couples to the surface at the plurality of anchoring points; and
a catheter fitting, coupled to the base, that secures the catheter in the channel.

2. The device of claim 1, wherein the base includes an underside that is coupleable to the surface and wherein the channel is defined by the underside of the base.

3. The device of claim 1, wherein the longitudinal opening is an open slot, such that the channel has a non-enclosed cross-section.

4. The device of claim 3, wherein the channel has an approximately semi-circular cross-section.

5. The device of claim 1, wherein the longitudinal opening is a slit, such that the channel has an enclosed cross-section that is openable at the slit.

6. The device of claim 5, wherein the channel has an approximately circular cross-section.

7. The device of claim 1, wherein the channel is non-parallel with respect to the underside of the base.

8. The device of claim 1, wherein the plurality of anchoring points include at least two anchoring points that are coupleable to the surface that substantially oppose on another across the catheter insertion site.

9. The device of claim 8, wherein the plurality of anchoring points includes at least one anchoring point adapted to lie proximal to the catheter insertion site and at least one anchoring point adapted to lie distal to the catheter insertion site.

10. The device of claim 8, wherein the plurality of anchoring points includes at least two substantially opposing anchoring points adapted to lie lateral to the catheter insertion site.

11. device of claim 8, wherein the plurality of anchoring points includes at least three anchoring points that are adapted to be distributed around the insertion site.

12. The device of Claim 11, wherein the frame includes an enclosed perimeter adapted to surround the catheter insertion site.

13. The device of claim 12, wherein the frame is substantially elliptical.

14. The device of claim 1, wherein the catheter fitting conforms to the received portion of the catheter and is slidingly engaged with the base such that the catheter fitting selectively operates between:
an unlocked position, in which the catheter fitting does not secure the catheter in the channel; and
a locked position, in which the catheter fitting conforms to and secures the catheter in the channel.

15. The device of claim 1, wherein the catheter fitting includes a flexible fitting that snap fits to the received portion of the catheter.

16. The device of claim 1, wherein the base is adapted to couple to the surface with an attachment element.

17. The device of claim 16, wherein the base is adapted to couple to the surface with an adhesive attachment element.

18. The device of claim 1, wherein the base further comprises a tubing router that is adapted to secure and route tubing associated with the catheter.

19. The device of claim 18, wherein the tubing router includes an approximately 180-degree turn.

20. A method for stabilizing an inserted catheter penetrating a surface at an insertion site, comprising:
providing a base having an underside that defines a channel with a longitudinal opening;
positioning the base over the catheter;
receiving a portion of the catheter in the channel through the longitudinal opening;
securing the catheter within the channel; and
coupling the base to the surface at a plurality of anchoring points distributed around the insertion site.

21. The method of claim 20, wherein receiving a portion of the catheter includes receiving a catheter hub.

22. The method of claim 20, wherein securing the catheter within the channel includes pressing a conformable fitting over the received portion of the catheter.

23. The method of claim 20, wherein coupling the base to the surface includes taping the base to the surface.

24. The method of claim 20, wherein coupling the base to the surface includes coupling the base to the surface at at least two substantially opposing anchoring points.

25. The method of claim 20, further including routing tubing associated with the catheter in a tubing router.

26. The method of claim 25, wherein routing tubing includes routing tubing through an approximately 180-degree turn.

27. A catheter stabilization device for stabilizing an inserted catheter penetrating a surface at an insertion site, comprising:
a base having an underside that is coupleable to the surface, wherein the base includes:
a channel defining a longitudinal opening that receives a portion of the catheter; and
a frame that forms at least a partial perimeter around the catheter insertion site with a plurality of anchoring points;
wherein the base couples to the surface at the plurality of anchoring points; and
a catheter fitting, coupled to the base, that conforms to the received portion of the catheter and is slidingly engaged with the base such that the fitting selectively operates between:
an unlocked position, in which the catheter fitting does not secure the catheter in the channel; and
a locked position, in which the catheter fitting snap fits to the catheter thereby securing the catheter in the channel; and
a tubing router, coupled to the base, that secures and routes tubing associated with the catheter through an approximately 180-degree turn.
